(19) United States
(12) Patent Application Publication
(10) Pub. No.: US 2023/0173271 A1
(43) Pub. Date: Jun. 8, 2023

(54) SENSOR-BASED TINNITUS TREATMENT SYSTEMS AND METHODS

(71) Applicant: Advanced Bionics AG, Staefa (CH)

(72) Inventors: Leonid M. Litvak, Los Angeles, CA (US); Stefan Launer, Zurich (CH); Elizabeth Galster, Studio City, CA (US); Hannah A. Glick, Ventura, CA (US)

(21) Appl. No.: 17/923,833

(22) PCT Filed: May 21, 2021
(86) PCT No.: PCT/US2021/033698

§ 371 Date: Nov. 7, 2022

Related U.S. Application Data
(60) Provisional application No. 63/031,386, filed on May 28, 2020.

(51) Int. Cl. A61N 1/36

(52) U.S. Cl. CPC A61N 1/36038; A61N 1/361; A61N 1/36139

(57) ABSTRACT

An exemplary system includes an implantable stimulator configured to be implanted within a recipient and apply electrical stimulation configured to treat tinnitus within the recipient. The system further includes an implantable sensor configured to be implanted within the recipient and output first sensor data representative of a first property associated with the recipient. The system further includes an external sensor configured to be external to the recipient and output second sensor data representative of a second property associated with the recipient. The system further includes a controller communicatively coupled to the implant, the implantable sensor, and the external sensor. The controller is configured to receive the first and second sensor data, and control, based on the first and second sensor data, the electrical stimulation.

### RELATED APPLICATIONS

The present application claims priority to U.S. Provisional Pat. Application No. 63/031,386, filed May 28, 2020, the contents of which are hereby incorporated by reference in their entirety.

### BACKGROUND INFORMATION

Tinnitus is often a symptom of noise-induced hearing loss but may also result from ototoxic drugs and diseases such as Meniere’s, vascular conditions, ear infections, and brain tumors. Although the mechanisms of tinnitus are not well understood, treatment may include behavioral therapy, masking, and drug treatments. However, no treatment has heretofore been shown to fully address tinnitus for a majority of sufferers.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary configuration of an implantable stimulator configured to treat tinnitus.

FIG. 2 illustrates another exemplary configuration of the implantable stimulator of FIG. 1.

FIG. 3 illustrates an exemplary cochlear implant system.

FIG. 4 shows an exemplary configuration of the cochlear implant system of FIG. 3.

FIG. 5 shows another exemplary configuration of the cochlear implant system of FIG. 3.

FIG. 6 illustrates an exemplary method to treat tinnitus with an implantable stimulator.

FIG. 7 illustrates an exemplary computing device.

### DETAILED DESCRIPTION

Sensor-based tinnitus treatment with a tinnitus treatment system is described herein. To illustrate, a tinnitus treatment system may include an implantable stimulator configured to be implanted within a recipient and apply electrical stimulation configured to treat tinnitus within the recipient. The system may further include an implantable sensor configured to be implanted within the recipient and output first sensor data representative of a first property associated with the recipient and an external sensor configured to be external to the recipient and output second sensor data representative of a second property associated with the recipient. The system may further include a controller communicatively coupled to the implant and the implantable sensor and the external sensor. The controller may be configured to receive the first and second sensor data, and control, based on the first and second sensor data, the electrical stimulation.

The systems and methods described herein may allow for treatment of tinnitus with an implantable stimulator based on sensor data received from one or more implantable sensors and/or one or more external sensors. For instance, a tinnitus treatment system as described herein may improve tinnitus treatment based on measured properties associated with the recipient. As such properties change, the treatment may be modulated for more effective reduction of tinnitus. Additionally, the tinnitus treatment system may receive feedback from the recipient and further improve tinnitus treatment based on the feedback. These and other advantages and benefits of the present systems and methods are described in more detail herein.

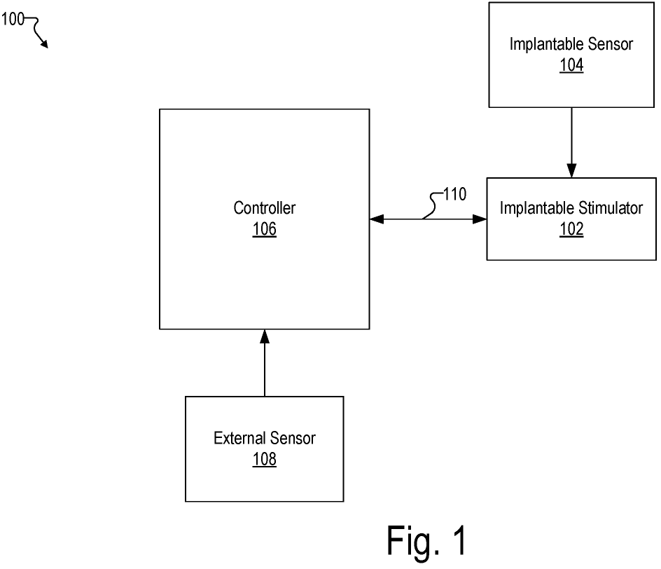

FIG. 1 illustrates an exemplary tinnitus treatment system 100 configured to be used by a recipient. As shown, tinnitus treatment system 100 includes an implantable stimulator 102 and an implantable sensor 104 communicatively coupled to implantable stimulator 102. Tinnitus treatment system 100 further includes a controller 106 and an external sensor 108 communicatively coupled to controller 106. Controller 106 is further configured to be communicatively coupled to implantable stimulator 102 by way of a communication link 110.

Implantable stimulator 102 may be implemented by any suitable implantable medical device that is configured to be implanted in a recipient and provide stimulation (e.g., electrical stimulation) to treat tinnitus within the recipient. For example, implantable stimulator 102 may be implemented by a cochlear implant, a deep brain stimulator, a neurostimulator, and/or any other suitable stimulator configured to provide stimulation to stimulation sites within the recipient.

Implantable sensor 104 may be implemented by any suitable sensor or plurality of sensors configured to be implanted in the recipient. The sensor or sensors may be configured to measure one or more properties and output sensor data representative of the one or more properties. For instance, implantable sensor 104 may measure any suitable property associated with the recipient such as an electroencephalograph (EEG) reading, a component of an EEG reading (e.g., an alpha brain wave reading, a beta brain wave reading, a theta brain wave reading, etc.), a stress level, an otoacoustic emission level, a body temperature, a heart rate, an oxygen level, a blood pressure level, and/or any other such property associated with the recipient. While FIG. 1 shows implantable sensor 104 communicatively coupled to implantable stimulator 102, implantable sensor 104 may additionally or alternatively be communicatively coupled directly to controller 106.

External sensor 108 may be implemented by any suitable sensor or plurality of sensors configured to be placed external to or at least partially external to the recipient. The sensor or sensors may be configured to measure one or more properties and output sensor data representative of the one or more properties. For example, external sensor 108 may measure any suitable property associated with the recipient such as a stress level, an otoacoustic emission level, a body temperature, a heart rate, an oxygen level, a blood pressure level, a skin conductance level, a property associated with an environment of the recipient (e.g., a sound pressure level), a movement of the recipient, and/or any other such property associated with the recipient.

Controller 106 may be configured to interface with (e.g., control and/or receive data from) implantable stimulator 102. For example, controller 106 may transmit commands (e.g., stimulation parameters and/or other types of operating parameters in the form of data words included in a forward telemetry sequence) to implantable stimulator 102 by way of communication link 110. Controller 106 may additionally or alternatively provide operating power to implantable stimulator 102 by transmitting one or more power signals to implantable stimulator 102 by way of communication link 110. Controller 106 may additionally or alternatively receive data from implantable stimulator 102 by way of communication link 110. Controller 106 may additionally receive data from implantable sensor 104 by way of implantable stimulator 102 and/or receive data directly from implantable sensor 104 by way of communication link 110 and/or a separate communication link (not shown). Communication link 110 may be implemented by any suitable number of wired and/or wireless bidirectional and/or unidirectional links.

Controller 106 may be further configured to receive sensor data from implantable sensor 104 and external sensor 108. Controller 106 may control, based on the received sensor data, the electrical stimulation that is to be applied by implantable stimulator 102 to treat the tinnitus within the recipient. In this manner, controller 106 may control (e.g., start, stop, increase, decrease, change, modulate, adjust, etc.) the electrical stimulation based on measurements of properties associated with the recipient. For example, implantable sensor 104 may include one or more electrodes configured to obtain EEG measurements. Based on the EEG measurements, controller 106 may control the electrical stimulation accordingly. For instance, a change in the EEG measurements (e.g., an alpha brain wave measurement) may indicate a change in stress level of the recipient, such as an increase in stress for the recipient. In response, controller 106 may modulate the electrical stimulation, such as change an amplitude of the electrical stimulation corresponding to the change in stress level.

As another example, external sensor 108 may include a microphone configured to measure a sound pressure level of an environment of the recipient. For instance, the microphone may be implemented by a microphone of a hearing aid, a microphone of an external device (e.g., a phone), or any other suitable microphone. The microphone may measure the sound pressure level and provide the measurements to controller 106, which may control the electrical stimulation in response to the sound pressure level of the environment of the recipient. For instance, the microphone may detect an increase in the sound pressure level of the environment of the recipient. In response, controller 106 may modulate the electrical stimulation, such as change an amplitude of the electrical stimulation corresponding to the increase in sound pressure level.

In some examples, controller 106 may control the electrical stimulation based on a combination of such properties measured by implantable sensor 104 and/or external sensor 108. For instance, the modulation of the electrical stimulation may be based on magnitudes and types of changes in the properties associated with the recipient. The modulation of the electrical stimulation may include controlling various parameters (e.g., frequency, pulse width, amplitude, burst pattern, stimulation site selection, etc.) of the electrical stimulation to be applied within the recipient. For example, a change in an EEG measurement combined with a change in sound pressure level may lead to a different change in the electrical stimulation than a change in either property alone. As another example, a change in body temperature and heart rate of the recipient along with a change in EEG measurement may provide a stronger indication of a change in stress level, and accordingly, controller 106 may change the electrical stimulation with a greater magnitude than in response to a change in only one of such properties. Any suitable combination of any such properties associated with the recipient may be used to determine modulations in the electrical stimulation by controller 106.

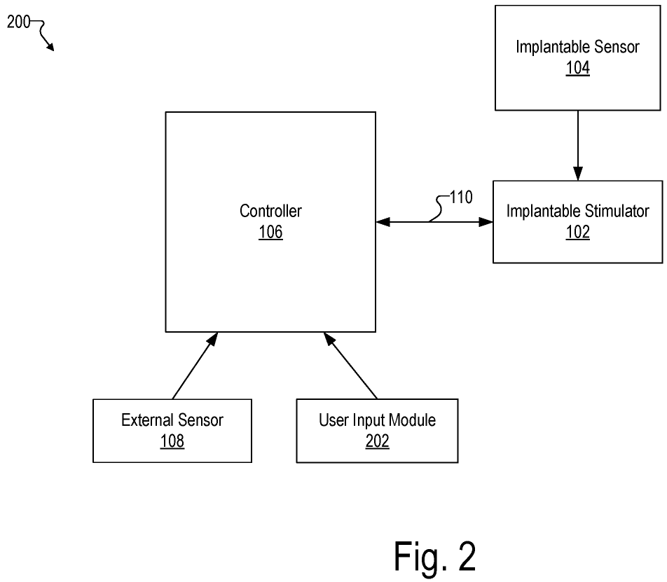

FIG. 2 illustrates another exemplary tinnitus treatment system 200 configured to be used by a recipient. Similar to tinnitus treatment system 100, tinnitus treatment system 200 includes implantable stimulator 102, implantable sensor 104, controller 106, external sensor 108, and communication link 110. Tinnitus treatment system 200 further includes a user input module 202.

User input module 202 may include any suitable module configured to receive input from a user (e.g., the recipient of tinnitus treatment system 200). For example, user input module 202 may be implemented on a device that includes controller 106 (e.g., buttons or other user interface on a hearing device, a component of a cochlear implant system, a component of a deep brain stimulation system, etc.) and/or on an external device communicatively coupled to controller 106 (e.g., an application running on a smartphone, tablet, computer, etc.). The input provided by the recipient may indicate whether tinnitus perceived by the recipient is improving, worsening, or is not noticeably changed. For instance, a change in a property associated with the recipient may result in a change in the perceived tinnitus. The recipient may provide input indicating whether the change in the perceived tinnitus is an improvement or a worsening of the tinnitus. Additionally, a change in the electrical stimulation may also result in a change in the perceived tinnitus. The recipient may again provide input indicating whether the change in the perceived tinnitus is an improvement or a worsening of the tinnitus. In some instances, the perceived tinnitus may improve or worsen absent a change in the electrical stimulation or in any of the properties measured by implantable sensor 104 or external sensor 108. The recipient may still provide input indicating the change in the perceived tinnitus and controller 106 may adjust the electrical stimulation provided by implantable stimulator 102. In this manner, tinnitus treatment system 200 may provide a closed loop system to improve treatment of the tinnitus.

As an example, implantable sensor 104 and/or external sensor 108 may detect a change in a property associated with the user (e.g., an internal microphone that detects a change in an otoacoustic emission level or any other suitable sensor detecting a change in any other suitable property). The detected change in the property may correlate with a change in the perceived tinnitus. The recipient may provide user input via user input module 202 to indicate whether the tinnitus has improved or worsened. In response, controller 106 may modulate the electrical stimulation provided by implantable stimulator 102 to treat the tinnitus. The recipient may then indicate via user input module 202 whether the modulation of the electrical stimulation has improved or worsened the perceived tinnitus. If the recipient indicates that the tinnitus has improved, controller 106 may direct implantable stimulator 102 to continue providing the electrical stimulation or controller 106 may increase the modulation of the electrical stimulation. If the recipient indicates that the tinnitus has worsened, controller 106 may decrease, invert, or otherwise change the modulation of the electrical stimulation. In this manner, tinnitus treatment system 200 may receive user input based on any suitable property or combination of properties measured by implantable sensor 104 and/or external sensor 108 to improve treatment of tinnitus in the recipient.

Additionally, based on the user input provided by the recipient and the changes in the properties measured by implantable sensor 104 and/or external sensor 108, tinnitus treatment system 200 may use one or more machine learning algorithms to determine effects of one or more properties on the tinnitus perceived by the recipient. Based on such determinations, tinnitus treatment system 200 may control the electrical stimulation for treating the tinnitus accordingly. For example, if for a threshold percentage of the time, when tinnitus treatment system 200 detects an increase in sound pressure level and a specific change in an EEG measurement the recipient indicates a worsening of the tinnitus, tinnitus treatment system 200 may determine that such a combination of changes in properties triggers a worsening in the tinnitus. As a result, tinnitus treatment system 200 may control the electrical stimulation to improve the perceived tinnitus when the combination of the increase in sound pressure level and the specific change in the EEG measurement is detected, without the recipient having to indicate that the tinnitus has worsened.

Further, based on the user input provided by the recipient and the changes in the electrical stimulation provided by implantable stimulator 102, tinnitus treatment system 200 may use machine learning algorithms to determine effects of magnitudes and types of electrical stimulation modulations on the tinnitus perceived by the recipient. For instance, the electrical stimulation may include particular amplitudes at particular stimulation sites. Changes to the electrical stimulation may include changes in amplitudes, stimulation sites, or both. Tinnitus treatment system 200 may determine, based on the user input, what types of changes improve, worsen, or do not noticeably change the perceived tinnitus. Additionally, tinnitus treatment system 200 may be able to further correlate types of changes in the electrical stimulation with the types of changes in the measured properties based on the user input. For example, electrical stimulation to specific stimulation sites may result in improved perceived tinnitus when a particular property associated with the recipient (e.g., if the recipient is in a car or a plane, which may be measured by an accelerometer of external sensor 108 or any other such correlation between particular measured properties and particular electrical stimulation).

In some examples, user input module 202 may include one or more additional input options. For examples, user input module 202 may be configured to receive input indicating qualitative differences in the perceived tinnitus, a desired rate of change in the modulation of the electrical stimulation, a desired type of change in the electrical stimulation and/or a particular setting of electrical stimulation, or any other suitable input to control the electrical stimulation provided to treat the tinnitus.

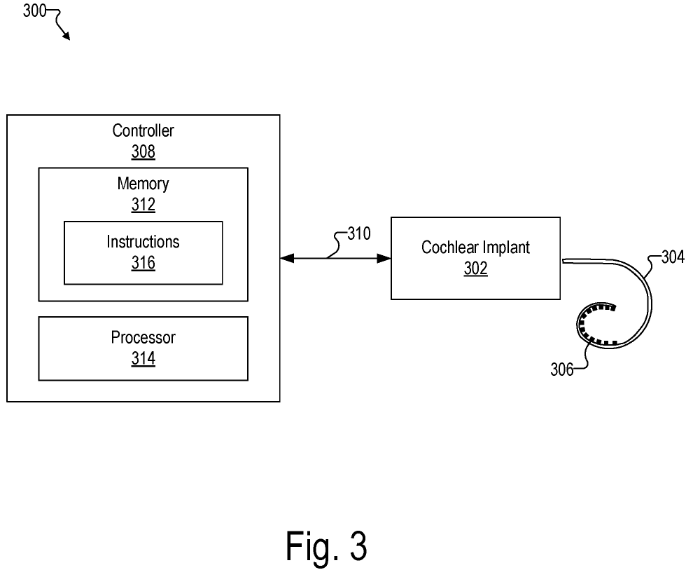

FIG. 3 illustrates an exemplary cochlear implant system 300, which may be an example implementation of tinnitus treatment system 100. As shown, cochlear implant system 300 includes a cochlear implant 302, an electrode lead 304 physically coupled to cochlear implant 302 and having an array of electrodes 306, and a controller 308 (e.g., an implementation of controller 106) configured to be communicatively coupled to cochlear implant 302 by way of a communication link 310 (e.g., an implementation of communication link 110).

The cochlear implant system 300 shown in FIG. 3 is unilateral (i.e., associated with only one ear of the recipient). Alternatively, a bilateral configuration of cochlear implant system 300 may include separate cochlear implants and electrode leads for each ear of the recipient. In the bilateral configuration, controller 308 may be implemented by a single controller configured to interface with both cochlear implants or by two separate controllers each configured to interface with a different one of the cochlear implants.

Cochlear implant 302 may be implemented by any suitable type of implantable stimulator. For example, cochlear implant 302 may be implemented by an implantable cochlear stimulator. Additionally or alternatively, cochlear implant 302 may be implemented by a brainstem implant and/or any other type of device that may be implanted within the recipient and configured to apply electrical stimulation to one or more stimulation sites located along an auditory pathway of the recipient.

In some examples, cochlear implant 302 may, in addition to generating electrical stimulation for treating tinnitus, be configured to generate electrical stimulation representative of an audio signal processed by controller 308 in accordance with one or more stimulation parameters transmitted to cochlear implant 302 by controller 308. Cochlear implant 302 may be further configured to apply the electrical stimulation to one or more stimulation sites (e.g., one or more intracochlear locations) within the recipient by way of one or more electrodes 306 on electrode lead 304. In some examples, cochlear implant 302 may include a plurality of independent current sources each associated with a channel defined by one or more of electrodes 306. In this manner, different stimulation current levels may be applied to multiple stimulation sites simultaneously by way of multiple electrodes 306.

Cochlear implant 302 may additionally or alternatively be configured to generate, store, and/or transmit data. For example, cochlear implant may use one or more electrodes 306 to record one or more signals (e.g., one or more voltages, impedances, evoked responses within the recipient, and/or other measurements) and transmit, by way of communication link 310, data representative of the one or more signals to controller 308. In some examples, this data is referred to as back telemetry data.

Electrode lead 304 may be implemented in any suitable manner. For example, a distal portion of electrode lead 304 may be pre-curved such that electrode lead 304 conforms with the helical shape of the cochlea after being implanted. Electrode lead 304 may alternatively be naturally straight or of any other suitable configuration.

In some examples, electrode lead 304 includes a plurality of wires (e.g., within an outer sheath) that conductively couple electrodes 306 to one or more current sources within cochlear implant 302. For example, if there are n electrodes 306 on electrode lead 304 and n current sources within cochlear implant 302, there may be n separate wires within electrode lead 304 that are configured to conductively connect each electrode 306 to a different one of the n current sources. Exemplary values for n are 8, 12, 16, or any other suitable number.

Electrodes 306 are located on at least a distal portion of electrode lead 304. In this configuration, after the distal portion of electrode lead 304 is inserted into the cochlea, electrical stimulation may be applied by way of one or more of electrodes 306 to one or more intracochlear locations. One or more other electrodes (e.g., including a ground electrode, not explicitly shown) may also be disposed on other parts of electrode lead 304 (e.g., on a proximal portion of electrode lead 304) to, for example, provide a current return path for stimulation current applied by electrodes 306 and to remain external to the cochlea after the distal portion of electrode lead 304 is inserted into the cochlea. Additionally or alternatively, a housing of cochlear implant 302 may serve as a ground electrode for stimulation current applied by electrodes 306.

Controller 308 may be configured to interface with (e.g., control and/or receive data from) cochlear implant 302. For example, controller 308 may transmit commands (e.g., stimulation parameters and/or other types of operating parameters in the form of data words included in a forward telemetry sequence) to cochlear implant 302 by way of communication link 310. Controller 308 may additionally or alternatively provide operating power to cochlear implant 302 by transmitting one or more power signals to cochlear implant 302 by way of communication link 310. Controller 308 may additionally or alternatively receive data from cochlear implant 302 by way of communication link 310. Communication link 310 may be implemented by any suitable number of wired and/or wireless bidirectional and/or unidirectional links.

As shown, controller 308 includes a memory 312 and a processor 314 configured to be selectively and communicatively coupled to one another. In some examples, memory 312 and processor 314 may be distributed between multiple devices and/or multiple locations as may serve a particular implementation.

Memory 312 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc. Exemplary volatile storage media include, but are not limited to, RAM (e.g., dynamic RAM).

Memory 312 may maintain (e.g., store) executable data used by processor 314 to perform one or more of the operations described herein. For example, memory 312 may store instructions 316 that may be executed by processor 314 to perform any of the operations described herein. Instructions 316 may be implemented by any suitable application, program (e.g., sound processing program), software, code, and/or other executable data instance. Memory 312 may also maintain any data received, generated, managed, used, and/or transmitted by processor 314.

Processor 314 may be configured to perform (e.g., execute instructions 316 stored in memory 312 to perform) various operations with respect to cochlear implant 302.

To illustrate, processor 314 may be configured to control an operation of cochlear implant 302. For example, processor 314 may receive an audio signal (e.g., by way of a microphone communicatively coupled to controller 308, a wireless interface (e.g., a Bluetooth interface), and/or a wired interface (e.g., an auxiliary input port)). Processor 314 may process the audio signal in accordance with a sound processing program (e.g., a sound processing program stored in memory 312) to generate appropriate stimulation parameters. Processor 314 may then transmit the stimulation parameters to cochlear implant 302 to direct cochlear implant 302 to apply electrical stimulation representative of the audio signal to the recipient.

In some implementations, processor 314 may also be configured to apply acoustic stimulation to the recipient. For example, a receiver (also referred to as a loudspeaker) may be optionally coupled to controller 308. In this configuration, processor 314 may deliver acoustic stimulation to the recipient by way of the receiver. The acoustic stimulation may be representative of an audio signal (e.g., an amplified version of the audio signal), configured to elicit an evoked response within the recipient, and/or otherwise configured. In configurations in which processor 314 is configured to both deliver acoustic stimulation to the recipient and direct cochlear implant 302 to apply electrical stimulation to the recipient, cochlear implant system 300 may be referred to as a bimodal hearing system and/or any other suitable term.

Processor 314 may be additionally or alternatively configured to receive and process data generated by cochlear implant 302. For example, processor 314 may receive data representative of a signal recorded by cochlear implant 302 using one or more electrodes 306 and, based on the data, adjust one or more operating parameters of controller 308. Additionally or alternatively, processor 314 may use the data to perform one or more diagnostic operations with respect to cochlear implant 302 and/or the recipient.

Other operations may be performed by processor 314 as may serve a particular implementation. In the description provided herein, any references to operations performed by controller 308 and/or any implementation thereof may be understood to be performed by processor 314 based on instructions 316 stored in memory 312.

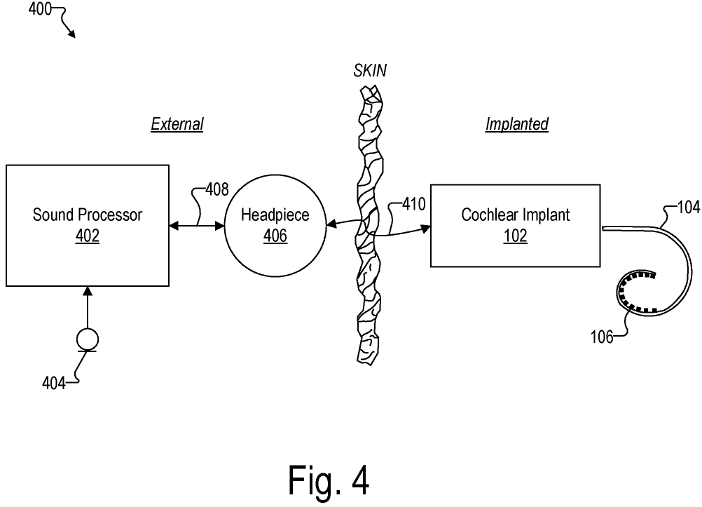

Controller 308 may be implemented by one or more devices configured to interface with cochlear implant 302. To illustrate, FIG. 4 shows an exemplary configuration 400 of cochlear implant system 300 in which controller 308 is implemented by a sound processor 402 configured to be located external to the recipient. In configuration 400, sound processor 402 is communicatively coupled to a microphone 404 and to a headpiece 406 that are both configured to be located external to the recipient.

Sound processor 402 may be implemented by any suitable device that may be worn or carried by the recipient. For example, sound processor 402 may be implemented by a behind-the-ear (“BTE”) unit configured to be worn behind and/or on top of an ear of the recipient. Additionally or alternatively, sound processor 402 may be implemented by an off-the-ear unit (also referred to as a body worn device) configured to be worn or carried by the recipient away from the ear. Additionally or alternatively, at least a portion of sound processor 402 is implemented by circuitry within headpiece 406.

Microphone 404 is configured to detect one or more audio signals (e.g., that include speech and/or any other type of sound) in an environment of the recipient. Microphone 404 may be implemented in any suitable manner. For example, microphone 404 may be implemented by a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 402. Additionally or alternatively, microphone 404 may be implemented by one or more microphones in or on headpiece 406, one or more microphones in or on a housing of sound processor 402, one or more beam-forming microphones, and/or any other suitable microphone as may serve a particular implementation.

Headpiece 406 may be selectively and communicatively coupled to sound processor 402 by way of a communication link 408 (e.g., a cable or any other suitable wired or wireless communication link), which may be implemented in any suitable manner. Headpiece 406 may include an external antenna (e.g., a coil and/or one or more wireless communication components) configured to facilitate selective wireless coupling of sound processor 402 to cochlear implant 302. Headpiece 406 may additionally or alternatively be used to selectively and wirelessly couple any other external device to cochlear implant 302. To this end, headpiece 406 may be configured to be affixed to the recipient’s head and positioned such that the external antenna housed within headpiece 406 is communicatively coupled to a corresponding implantable antenna (which may also be implemented by a coil and/or one or more wireless communication components) included within or otherwise connected to cochlear implant 302. In this manner, stimulation parameters and/or power signals may be wirelessly and transcutaneously transmitted between sound processor 402 and cochlear implant 302 by way of a wireless communication link 410.

In configuration 400, sound processor 402 may receive an audio signal detected by microphone 404 by receiving a signal (e.g., an electrical signal) representative of the audio signal from microphone 404. Sound processor 402 may additionally or alternatively receive the audio signal by way of any other suitable interface as described herein. Sound processor 402 may process the audio signal in any of the ways described herein and transmit, by way of headpiece 406, stimulation parameters to cochlear implant 302 to direct cochlear implant 302 to apply electrical stimulation representative of the audio signal to the recipient.

In an alternative configuration, sound processor 402 may be implanted within the recipient instead of being located external to the recipient. In this alternative configuration, which may be referred to as a fully implantable configuration of cochlear implant system 300, sound processor 402 and cochlear implant 302 may be combined into a single device or implemented as separate devices configured to communicate one with another by way of a wired and/or wireless communication link. In a fully implantable implementation of cochlear implant system 300, headpiece 406 may not be included and microphone 404 may be implemented by one or more microphones implanted within the recipient, located within an ear canal of the recipient, and/or external to the recipient.

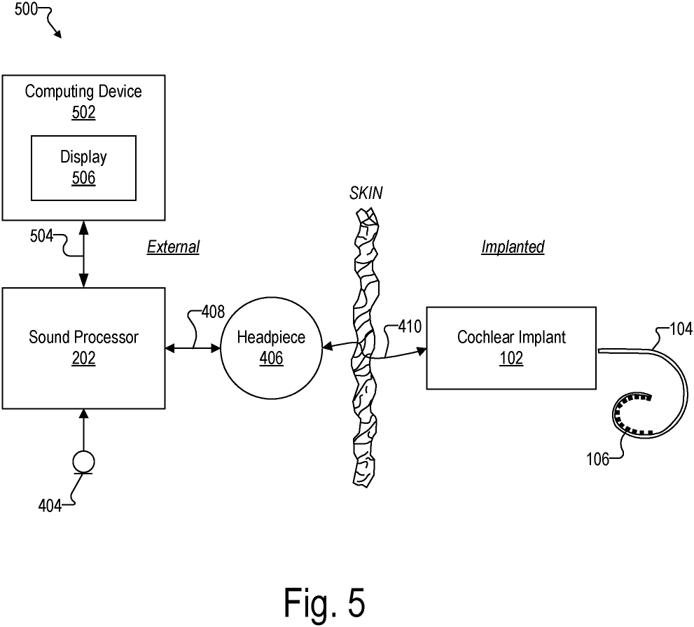

FIG. 5 shows an exemplary configuration 500 of cochlear implant system 300 in which controller 308 is implemented by a combination of sound processor 402 and a computing device 502 configured to communicatively couple to sound processor 402 by way of a communication link 504, which may be implemented by any suitable wired or wireless communication link.

Computing device 502 may be implemented by any suitable combination of hardware and software. To illustrate, computing device 502 may be implemented by a mobile device (e.g., a mobile phone, a laptop, a tablet computer, etc.), a desktop computer, and/or any other suitable computing device as may serve a particular implementation. As an example, computing device 502 may be implemented by a mobile device configured to execute an application (e.g., a “mobile app”) that may be used by a user (e.g., the recipient, a clinician, and/or any other user) to control one or more settings of sound processor 402 and/or cochlear implant 302 and/or perform one or more operations (e.g., diagnostic operations) with respect to data generated by sound processor 402 and/or cochlear implant 302.

In some examples, computing device 502 may be configured to control an operation of cochlear implant 302 by transmitting one or more commands to cochlear implant 302 by way of sound processor 402. Likewise, computing device 502 may be configured to receive data generated by cochlear implant 302 by way of sound processor 402. Alternatively, computing device 502 may interface with (e.g., control and/or receive data from) cochlear implant 302 directly by way of a wireless communication link between computing device 502 and cochlear implant 302. In some implementations in which computing device 502 interfaces directly with cochlear implant 302, sound processor 402 may or may not be included in cochlear implant system 300.

Computing device 502 is shown as having an integrated display 506. Display 506 may be implemented by a display screen, for example, and may be configured to display content generated by computing device 502. Additionally or alternatively, computing device 502 may be communicatively coupled to an external display device (not shown) configured to display the content generated by computing device 502.

In some examples, computing device 502 represents a fitting device configured to be selectively used (e.g., by a clinician) to fit sound processor 402 and/or cochlear implant 302 to the recipient. In these examples, computing device 502 may be configured to execute a fitting program configured to set one or more operating parameters of sound processor 402 and/or cochlear implant 302 to values that are optimized for the recipient. As such, in these examples, computing device 502 may not be considered to be part of cochlear implant system 300. Instead, computing device 502 may be considered to be separate from cochlear implant system 300 such that computing device 502 may be selectively coupled to cochlear implant system 300 when it is desired to fit sound processor 402 and/or cochlear implant 302 to the recipient.

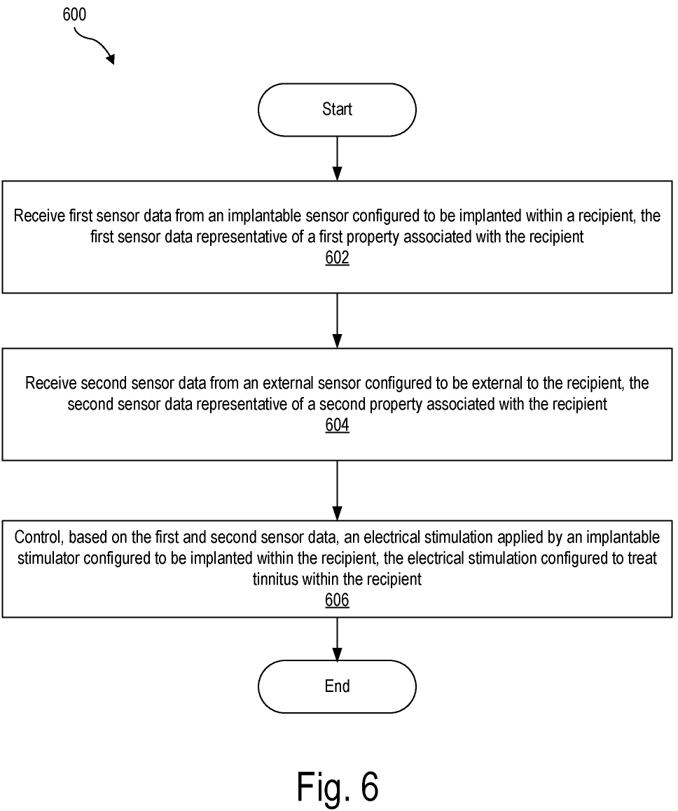

FIG. 6 illustrates an exemplary method 600. The operations shown in FIG. 6 may be performed by controller 106 and/or any implementation thereof. While FIG. 6 illustrates exemplary operations according to one embodiment, other embodiments may omit, add to, reorder, and/or modify any of the operations shown in FIG. 6.

In operation 602, a controller receives first sensor data from an implantable sensor configured to be implanted within a recipient, the first sensor data representative of a first property associated with the recipient. Operation 602 may be performed in any of the ways described herein.

In operation 604, the controller receives second sensor data from an external sensor configured to be external to the recipient, the second sensor data representative of a second property associated with the recipient. Operation 604 may be performed in any of the ways described herein.

In operation 606, the controller controls, based on the first and second sensor data, an electrical stimulation applied by an implantable stimulator configured to be implanted within the recipient, the electrical stimulation configured to treat tinnitus within the recipient. Operation 606 may be performed in any of the ways described herein.

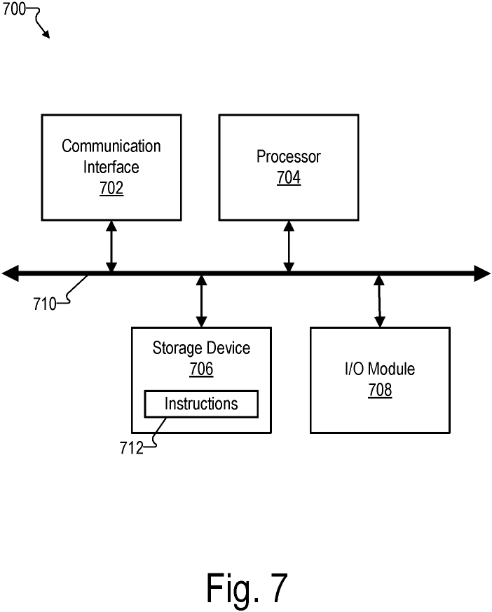

FIG. 7 illustrates an exemplary computing device 700 that may be specifically configured to perform one or more of the processes described herein. Any of the systems, units, computing devices, and/or other components described herein may be implemented by computing device 700.

As shown in FIG. 7, computing device 700 may include a communication interface 702, a processor 704, a storage device 706, and an input/output (“I/O”) module 708 communicatively connected one to another via a communication infrastructure 710. While an exemplary computing device 700 is shown in FIG. 7, the components illustrated in FIG. 7 are not intended to be limiting. Additional or alternative components may be used in other embodiments. Components of computing device 700 shown in FIG. 7 will now be described in additional detail.

Communication interface 702 may be configured to communicate with one or more computing devices. Examples of communication interface 702 include, without limitation, a wired network interface (such as a network interface card), a wireless network interface (such as a wireless network interface card), a modem, an audio/video connection, and any other suitable interface.

Processor 704 generally represents any type or form of processing unit capable of processing data and/or interpreting, executing, and/or directing execution of one or more of the instructions, processes, and/or operations described herein. Processor 704 may perform operations by executing computer-executable instructions 712 (e.g., an application, software, code, and/or other executable data instance) stored in storage device 706.

Storage device 706 may include one or more data storage media, devices, or configurations and may employ any type, form, and combination of data storage media and/or device. For example, storage device 706 may include, but is not limited to, any combination of the non-volatile media and/or volatile media described herein. Electronic data, including data described herein, may be temporarily and/or permanently stored in storage device 706. For example, data representative of computer-executable instructions 712 configured to direct processor 704 to perform any of the operations described herein may be stored within storage device 706. In some examples, data may be arranged in one or more databases residing within storage device 706.

I/O module 708 may include one or more I/O modules configured to receive user input and provide user output. I/O module 708 may include any hardware, firmware, software, or combination thereof supportive of input and output capabilities. For example, I/O module 708 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 708 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 708 is configured to provide graphical data to a display for presentation to a user. The graphical data may be representative of one or more graphical user interfaces and/or any other graphical content as may serve a particular implementation.

In some examples, any of the facilities described herein may be implemented by or within one or more components of computing device 700. For example, one or more computer readable instructions 712 (e.g., an application) residing within storage device 706 may be configured to direct an implementation of processor 704 to perform one or more operations or functions associated with processor 314 of system 300. Likewise, memory 312 of system 300 may be implemented by or within an implementation of storage device 706.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
an implantable stimulator configured to be implanted within a recipient and apply electrical stimulation configured to treat tinnitus within the recipient;
an implantable sensor configured to be implanted within the recipient and output first sensor data representative of a first property associated with the recipient;
an external sensor configured to be external to the recipient and output second sensor data representative of a second property associated with the recipient; and
a controller communicatively coupled to the implant, the implantable sensor, and the external sensor, the controller configured to:
receive the first and second sensor data, and
control, based on the first and second sensor data, the electrical stimulation.

2. The system of claim 1, wherein the first property includes one or more of an electroencephalograph (EEG) reading, an alpha brain wave reading, a stress level, an otoacoustic emission level, a body temperature, a heart rate, an oxygen level, or a blood pressure level.

3. The system of claim 1, wherein the second property includes one or more of a stress level, an otoacoustic emission level, a body temperature, a heart rate, an oxygen level, a blood pressure level, a skin conductance level, a sound pressure level in an environment of the recipient, or a movement of the recipient.

4. The system of claim 1, wherein:
the first property includes an EEG reading; and
the controlling of the electrical stimulation includes changing an amplitude of the electrical stimulation based on a change in a component of the EEG reading.

5. The system of claim 1, wherein:
the second property includes a sound pressure level of an environment of the recipient; and
the controlling of the electrical stimulation includes changing an amplitude of the electrical stimulation based on a change in the sound pressure level.

6. The system of claim 1, wherein:
the first property includes an EEG reading;
the second property includes a sound pressure level of an environment of the recipient; and
the controlling the electrical stimulation includes changing an amplitude of the electrical stimulation based on a change in at least one of a component of the EEG reading and the sound pressure level.

7. The system of claim 1, wherein:
the controller is further configured to receive user input indicative of an effect of the electrical stimulation on the tinnitus; and
the controlling the electrical stimulation is further based on the user input.

8. The system of claim 7, wherein the controller is configured to receive the user input by way of an interface communicatively coupled to an external device.

9. The system of claim 7, wherein the controller is further configured to determine, based on the first sensor data, the second sensor data, and the user input, an effect on the tinnitus of at least one of the first and the second property.

10. The system of claim 9, wherein the controller is further configured to control the electrical stimulation based on the determined effect.

11. The system of claim 1, wherein:
the implantable stimulator comprises a cochlear implant coupled to a lead having a plurality of electrodes; and
the cochlear implant is configured to apply the electrical stimulation by way of one or more of the plurality of electrodes.

12. The system of claim 11, wherein the controller is further configured to direct the cochlear implant to apply additional electrical stimulation to represent an audio signal to the recipient.

13. A system comprising:
a cochlear implant configured to be implanted within a recipient, the cochlear implant coupled to a lead having a plurality of electrodes configured to:
apply electrical stimulation configured to treat tinnitus within the recipient, and
apply additional electrical stimulation configured to represent an audio signal to the recipient;
an implantable sensor configured to be implanted within the recipient and output first sensor data representative of a first property associated with the recipient;
an external sensor configured to be external to the recipient and output second sensor data representative of a second property associated with the recipient; and
a controller communicatively coupled to the cochlear implant, the implantable sensor and the external sensor, the controller configured to:
receive the first and second sensor data, and
control, based on the first and second sensor data, the electrical stimulation.

14. The system of claim 13, wherein the first property includes one or more of an electroencephalograph (EEG) reading, an alpha brain wave reading, a stress level, an otoacoustic emission level, a body temperature, a heart rate, an oxygen level, or a blood pressure level.

15. The system of claim 13, wherein the second property includes one or more of a stress level, an otoacoustic emission level, a body temperature, a heart rate, an oxygen level, a blood pressure level, a skin conductance level, a sound pressure level in an environment of the recipient, or a movement of the recipient.

16. The system of claim 13, wherein:
the first property includes an EEG reading;
the second property includes a sound pressure level of an environment of the recipient; and
the controlling the electrical stimulation includes changing an amplitude of the electrical stimulation based on a change in at least one of a component of the EEG reading and the sound pressure level.

17. The system of claim 13, wherein:
the controller is further configured to receive user input indicative of an effect of the electrical stimulation on the tinnitus; and
the controlling the electrical stimulation is further based on the user input.

18. A method comprising:
receiving, by a controller, first sensor data from an implantable sensor configured to be implanted within a recipient, the first sensor data representative of a first property associated with the recipient;
receiving second sensor data from an external sensor configured to be external to the recipient, the second sensor data representative of a second property associated with the recipient; and
controlling, by the controller and based on the first and second sensor data, an electrical stimulation applied by an implantable stimulator configured to be implanted within the recipient, the electrical stimulation configured to treat tinnitus within the recipient.

19. The method of claim 18, wherein:
the first property includes an EEG reading;
the second property includes a sound pressure level of an environment of the recipient; and
the controlling the electrical stimulation includes changing an amplitude of the electrical stimulation based on a change in at least one of a component of the EEG reading and the sound pressure level.

20. The method of claim 18, further comprising receiving, by the controller, user input indicative of an effect of the electrical stimulation on the tinnitus; and
wherein the controlling the electrical stimulation is further based on the user input.
